(19) United States
(12) Patent Application Publication
(10) Pub. No.: US 2021/0186885 A1
(43) Pub. Date: Jun. 24, 2021

(54) POWDER COATING COMPOSITIONS FOR COATING PHARMACEUTICAL PELLETS

(71) Applicant: POWDER PHARMA COATING INC., SCARBOROUGH (CA)

(72) Inventors: Jingxu ZHU, LONDON (CA); Yingliang MA, London (CA); Kwok Yui CHOW, Mississauga (CA); Qingliang YANG, London (CA); Yunhan ZHANG, Thornhill (CA); Herman C. LAM, Scarborough (CA)

(21) Appl. No.: 16/944,639

(22) Filed: Jul. 31, 2020

Related U.S. Application Data
(63) Continuation of application No. 15/787,547, filed on Oct. 18, 2017, now abandoned.
(60) Provisional application No. 62/410,120, filed on Oct. 19, 2016.

(51) Int. Cl. A61K 9/50; A61P 9/00; A61P 11/06; A61P 25/08; A61P 29/00; A61K 9/16; A61K 31/4439; A61K 31/5415

(52) U.S. Cl. CPC A61K 9/5026; A61K 31/5415; A61K 9/5073; A61P 9/00; A61P 11/06; A61P 25/08; A61P 29/00; A61K 9/1611; A61K 9/1635; A61K 9/1641; A61K 9/1652; A61K 9/167; A61K 9/1682; A61K 9/501; A61K 9/5015; A61K 9/5031; A61K 9/5089; A61K 31/4439; A61K 9/5078

(57) ABSTRACT

The present disclosure provides powder coating compositions for pharmaceutical pellets which include one or more film forming polymers in powder form present in the composition in a range from about 1 to about 95% w/w. The compositions include one or more plasticizers in powder or liquid form present in the composition in quantity to lower the glass transition temperature of the coating composition to a temperature in a range from about 30 to 100° C. The compositions also include one or more one anti-static agents in powder or liquid form present in the composition in a range from about 0.1 to about 95% w/w as well as one or more flow enhancing agents in powder form present in the composition in a range from about 0.1 to about 25% w/w.

### FIELD

The present disclosure relates to powder coating compositions for coating pharmaceutical pellets.

### BACKGROUND

Orally administered pharmaceutical products such as tablets are typically coated for many different reasons, including anyone or combination of moisture protection, delayed release of the medicinally active component, targeted drug delivery, extended release, taste masking, taste modification, and aesthetic appeal, to mention a few reasons.

Tablets have been coated using pan coaters in which the tablet cores to the coating are typically sprayed in either powder or liquid form, or a combination of both. Electrostatic powder coating is a relatively new film coating technique for the manufacture of coated tablets to achieve a wide range of functions such as modified release, moisture protection, aesthetics and taste masking functions. It is an environmental friendly and cost effective method that can potentially replace the existing aqueous and solvent coating methods. Electrostatic powder coating using a pan coating apparatus was introduced in United States Patent Publication No. 2007/0128274.

In addition to tablets, another orally administered pharmaceutical product is made of pellets which are much smaller than tablets. These smaller pellets can be orally administered in pre-set dosage amounts such as pellets in filled hard gelatin capsules, or they can be compressed together with additional excipients to form larger tablets such that these tablets are made from the smaller pellets. Typically, the administration of oral pellets provide significant clinical benefits such as consistent bioavailability of modified release products and patient safety benefits compared to monolithic tablets such as reduction of dose dumping of extended released formulations. It would be very advantageous to be able to coat these individual pellets but for the coated pellets to be viable the resulting coating must be uniform and coating the entire pellet surface.

The inventors have noted that the same formulations used for powder coating of tablets alone are not adequate for multi-particulate (pellet) coating. Due to the increased surface area and reduced bulk density of the much smaller pellets compared to the larger tablets, the agglomeration tendency of pellets is increased during the coating process. The larger specific area associated with the smaller pellets provides a more favorable environment for pellets to adhere together and their lower bulk density prevents the agglomerated pellets from separating from each other, thereby resulting in unevenly coated pellets.

One of the reasons for agglomeration during coating is due to polymer film stickiness associated with the polymers used to form the coatings. For example, coating of oral pharmaceutical products is commonly conducted using a liquid coating process where a coating film is produced by concurrent deposition and drying of polymeric coating material. The film coat is generally non-sticky when it is not wet and the product temperature is not too high. However, since the glass transition temperature of the coating material is lowered in the presence of plasticiser(s) and solvent, the coat surface can become sticky if the solvent is not evaporated quickly or the coating temperature is too high relative to the glass transition temperature. This results in product agglomeration if the product is over wetted due to insufficient product movement in the coating pan and/or excessive spray rate of coating material and high coating temperature. In powder coating, the glass transition temperature of the coating material is also decreased to facilitate film forming. An excessive reduction of the glass transition temperature increases the tackiness of the film coating causing agglomeration. The agglomeration is particularly problematic for pellets versus tablets as the pellets have a much smaller inertia to break off from each other.

Another reason of agglomeration of solid oral products in coating is the presence of electrostatic charge. Electrostatic charged surfaces attract much stronger than non-charged surfaces because of the stronger electrostatic force of charged units than the non-specific van der Waal force of the non-charged units. This is particularly problematic for the much smaller pellets than it is for the much larger tablets since much lighter pellets when charged up can electrostatically bind to oppositely charged pellets which results in stronger bonding than associated with lower surface area to volume tablets.

As noted above, agglomeration must be avoided to produce a quality film in coating of pellets. Agglomeration causes coating surface defects, coating dissolution failure, and in-vivo performance issues. Agglomeration of tablets and pellets in pharmaceutical film coating is a common cause of product manufacturing failures and inter- and intra-batch-to-batch variations in product performance such as bioavailability and absorption characteristics.

Therefore, the formulations used for powder coating of small pellets must include not only the functional constituents which give the resulting coat with desired pharmaceutical properties, but also must include constituents which facilitate the production of uniform coatings on the pellets during the powder coating process.

Thus, it would be very advantageous to provide formulations for electrostatic spray powder coating of pellets which avoid the aforementioned limitations.

### SUMMARY

The present disclosure provides powder coating compositions for pharmaceutical pellets which include one or more film forming polymers in powder form present in the composition in a range from about 1 to about 95% w/w. The compositions include one or more plasticizers in powder or liquid form present in the composition in quantity to lower the glass transition temperature of the coating composition to a temperature in a range from about 30 to 100° C. The compositions also include one or more one anti-static agents in powder or liquid form present in the composition in a range from about 0.1 to about 95% w/w as well as one or more flow enhancing agents in powder form present in the composition in a range from about 0.1 to about 25% w/w.

The one or more film forming polymers may be present in the composition in a range from about 10 to about 70% w/w.

The one or more flow enhancing agents may be present in the composition in a range from about 0.25 to about 20% w/w.

The one or more flow enhancing agents may be present in the composition in a range from about 0.5 to about 3% w/w.

The one or more anti-static agents may be present in the composition in a range from about 1 to about 50% w/w.

The one or more plasticizers may include any one or combination of glycerol, propylene glycol, PEG 200 to 8000 grades, triacetin, diethyl phthalate (DEP), dibutyl phthalate (DBP), tributyl citrate (TBC), triethyl citrate (TEC), oleyl alcohol, castor oil, fractionated coconut oil, acetylated monoglycerides, glycerol monostearate. Plasticizers may also include low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol) and citrate ester-type plasticizers.

The one or more plasticizers may include any one or combination of ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, acetyl triethyl citrate and allyl glycolate.

The one or more anti-static agents may include common salts, carbon black, magnesium stearate, fumed silicate, magnesium trisilicate, glycerol monostearate, Kaolin, talc and a liquid plasticizer. The liquid plasticizer may include any one or combination of PEG 200 to 600, propylene glycol, glycerin, and triacetin. The common salts may include any one or combination of sodium chloride, calcium chloride, magnesium hydroxide, sodium carbonate, sodium bicarbonate, sodium phosphate, sodium citrate, sodium acetate, potassium acetate, potassium citrate, potassium chloride, and magnesium sulfate.

The plasticizer may be selected to lower the glass transition temperature of the coating composition to a temperature in a range from about 45 to 70° C.

The one or more flow enhancing agents may include any one or combination of calcium stearate, colloidal silicon dioxide, hydrogenate castor oil and microcrystalline cellulose, fumaric acid, glycerol behanate, glycerol monostearate, glycerol palmitostearate, leucine, magnesium stearate, medium chain triglyceride, myristic acid, palmitic acid, poloxamer, polyethylene glycol, potassium benzoate, sodium benzoate, sodium lauryl sulfate, sodium stearyl fumarate, starch, stearic acid, talc, hydrogenated vegetable oil and zinc stearate.

The one or more film forming polymers may be selected to exhibit any one or combination of a moisture barrier, immediate release, flavoring, taste modifying, and taste masking, and wherein the film forming polymer includes any one or combination of methylcellulose, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers.

The one or more film forming polymers may be selected to exhibit extended release and includes any one or combination of cellulose ether derivative, acrylic resin, a copolymer of acrylic acid and methacrylic acid esters with quaternary ammonium groups, a copolymer of acrylic acid and methacrylic acid esters, ethyl cellulose, and poly(meth)acrylate polymers that are not soluble in digestive fluids.

The one or more film forming polymers may be selected to exhibit extended release and includes any one or combination of polyethylene oxide (PEO), ethylene oxide-propylene oxide co-polymers, polyethylene-polypropylene glycol (e.g. poloxamer), carbomer, polyvinyl pyrrolidone (PVP), polyvinyl alcohol (PVA), hydroxyalkyl celluloses such as hydroxypropyl cellulose (HPC), hydroxypropyl methylcellulose, sodium carboxymethyl cellulose, methylcellulose, hydroxyethyl methylcellulose, hydroxypropyl methylcellulose, polyacrylates such as carbomer, polyacrylamides, alginic acid and its derivatives, starch and starch derivatives, gelatin that are soluble in digestive fluids.

The poly(meth)acrylate polymers that are not soluble in digestive fluids may include any one or combination of Eudragit® RS polymers, Eudragit® RL polymers, and EUDRAGIT® NE polymers.

The one or more film forming polymers may be selected to exhibit delayed release include any one or combination of cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups.

The methacrylic copolymers with carboxylic acid groups may include Eudragit® L30D, Eudragit® L100, Eudragit® FS30D, Eudragit® SI00, Acryl-EZE®.

The present disclosure provides coated pharmaceutical pellets having at least two coating layers, comprising:

a) a first coating applied directly on the drug pellets that has a protective function; and

b) a second coating on the first layer that has a release modification function.

The present disclosure also provides coated pharmaceutical pellets having at least three coating layers, comprising:

a) a first coating coated directly on the drug pellets that has a protective function;

b) a second coating to be coated on the first coating that has a sustained/controlled release function; and

c) a third coating to be coated on said second coating that has a delayed release function.

The present disclosure also provides coated pharmaceutical pellets having at least four coatings, comprising:

a) a first coating applied directly on the pharmaceutical pellets that includes a first drug component;

b) a second coating on the first layer that including a second drug component separate from the first drug component;

c) a third coating on the second coating that has a protective function;

d) a fourth coating on the third coating that has a release modification function.

In the embodiments above having one or more drug compositions in the first and/or second drug layers may comprise any one or combination of moisture sensitive drugs including aspirin, melbine, esomeprazole, vitamins; anti-inflammatory, antipyretic, anticonvulsant and/or analgesic agents including indomethacin, nimesulide, ibuprofen, fenoprofen calcium; cardiocirculatory system drugs including nifedipine, felodipine, nimodipine, nilvadipine, lacidipine, doxazosin and anti-asthma drugs including salbutamol.

In the embodiments above having a protective function may be comprise any one or combination of hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition including Opadry® AMB and Aminoalkyl methacrylate copolymers.

In the above embodiments the layer having a sustained/controlled release function may comprise any one or combination of water soluble, water insoluble and pH sensitive polymers including polyethylene oxide (PEO), ethylene oxide-propylene oxide co-polymers, polyethylene-polypropylene glycol, poloxamer, carbomer, polycarbophil, chitosan, polyvinyl pyrrolidone (PVP), polyvinyl alcohol (PVA), hydroxyalkyl celluloses including hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates including carbomer, polyacrylamides, polymethacrylamides, polyphosphazines, polyoxazolidines, polyhydroxyalkylcarboxylic acids, alginic acid and its derivatives including carrageenate alginates, ammonium alginate and sodium alginate, starch and starch derivatives, polysaccharides, carboxypolymethylene, polyethylene glycol, natural gums including gum guar, gum acacia, gum tragacanth, karaya gum and gum xanthan, povidone and gelatin; and

In the above embodiments the coating having a delayed release function may comprise any one or combination of, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups including Eudragit® L30D, Eudragit® L100, Eudragit® FS30D, Eudragit® SI00 and Acryl-EZE®.

The present disclosure provides coated pharmaceutical pellets having at least two coating layers, comprising a first coating directly on the drug pellets that has a first preselected functionality; and at least a second coating on the first coating that has a second preselected functionality different from the first preselected functionality.

The first preselected functionality may be either a protective coating or a drug containing coating, and wherein the second functionality is a release modification function.

The release modification layer may be selected to achieve delayed release of the drug pellet, or it may be selected to achieve sustained/controlled release layer to provide a preselected release profile.

A further understanding of the functional and advantageous aspects of the present disclosure can be realized by reference to the following detailed description and drawings.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1 is an example dissolution profile of coated pellets with taste-masking film forming polymer powder;

FIG. 2 is an example dissolution profile of coated pellets with extended-release film forming polymer powder; and

FIG. 3 is an example dissolution profile of coated pellets with delayed film forming polymer powder.

FIGS. 4A to 4D are examples of multilayer coated pellets with different coating materials, which;

FIG. 4A is a cross sectional view of an example of a drug pellet coated with the protective layer and release modification layer to achieve delayed release or sustained/controlled release layer or any other modified release profiles;

FIG. 4B is a cross sectional view of an example of a drug pellet coated with the protective layer, followed by the sustained/controlled release layer and then delayed release layer, achieving both delayed drug release and sustained/controlled drug release;

FIG. 4C is a cross sectional view of an example of a pellet core firstly loaded with a single drug layer, followed by the protective coating layer and release modification coating layer; and

FIG. 4D is a cross sectional view of an example of a pellet core firstly loaded with different drug layers, followed by the protective layer and release modification layer.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof” mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

As used herein the phrases pellets, beads and spheroids (hereinafter pellets) are interchangeable terms as used herein to refer to small spherical or close to spherical single particles or agglomerations of fine powders or granules of pharmaceutical ingredients. It will be noted pellets may not be spherical but could have other shapes, such as but not limited to cylindrical, cubical etc. Pellets can be coated or uncoated, depending on its end usage. The pellet size ranges for commercially available pharmaceutical uncoated pellets is typically in the range from about 100 to about 2000 μm (0.10 to 2.00 mm). As used herein, pellets have sizes in a range from about 50 to about 3,000 μm (microns) (0.30 to 3.00 mm), with a preferable size range being from about 100 to about 2,000 μm. Uncoated pellets are prepared using a variety of palletisation methods including, but not limited to, wet granulation, extrusion/spheronization, hot melt extrusion, fluidbed layering, or powder layering methods.

Defined quantities of coated or uncoated pellets are filled into capsules or compressed into tablets along with pharmaceutically acceptable excipients to produce a dosage unit for oral administration. Pellets can also be administered directly or dispersed in a liquid as an oral suspension for oral administration. The use of pellets is not limited to oral administration. For example, pellets can be mixed with a semisolid based composition, such as, but not limited to creams, for use as a topical product.

In contrast, pharmaceutical tablets for humans have sizes in a range from about 5 mm to about 25 mm in the longest dimension of round, oblong, oval or any other shapes.

As used herein the phrase “film forming polymers” refer to polymers that produce a physical, continuous film upon curing when used as a coating material for powder coating. The continuous film may or not may not contain a plasticizer. Film forming polymers together with other pharmaceutical agents are used to produce functional, cosmetic or a combination thereof, film coats for pharmaceutical products. One or more film forming polymer coatings can provide one or a combination of, but not limited to, the following characteristics: 1) moisture protection, e.g. moisture protective film coating of a tablet or pellets; 2) delayed release characteristics, e.g. enteric film coating so that a drug will not be released in the stomach before it reaches the upper intestine; 3) targeted drug delivery, e.g. a delayed pH sensitive film coating of a tablet or pellets to colonic delivery of a drug so that the drug will start releasing in the lower GI tract; 4) extended release, e.g. a sustained release film coating of a tablet or pellets to provide prolonged drug released at a constant rate for a period of time after drug administration where product is typically taken once or twice daily instead several times a day; 5) taste masking to prevent dissolution in the mouth, and similarly taste modifying agents in the coating; and 6) low dose coating, e.g. a small amount (low dose) of drug substance is embedded in the polymer coating of a low dose product. The present disclosure provides compositions used to improve content uniformity of low dose products.

As used herein the phrase “plasticizer” refers to additives that soften a polymer by lowering its glass transition temperature or reducing its crystallinity or melting temperature. For powder coatings, an appropriate level of a plasticizer allows the polymer/plasticizer material to coalesce to form a continuous polymeric film at a defined time and temperature. Plasticizers also refer to additives for polymers for imparting desired viscosity, flexibility, plasticity and any other physical properties to produce a suitable coating film that can withstand the mechanical handling forces in the film coating process, product transfer, and packaging and transportation.

As used herein the phrase “anti-static agents” refers to additives that help eliminate electrostatic charges generated on a surface of pellets or tablets. One mechanism of charge elimination is obtained by increasing the conductivity of the surface in the presence of an electro-conducting anti-static agent. Another charge elimination mechanism is the use of a hygroscopic anti-static agent so that the surface moisture on pellet or tablet enhances charge dispersion. Anti-static agents prevent powder particle adhesion to each other and to non-electrical bonded or poorly bonded surfaces.

As used herein the phrase “flow enhancing agents” refers to additives that improve the flowability of powders. A suitable flow enhancing agent enables effective bulk powder transfer to the electrostatic spray gun during powder coating process.

The present compositions have been developed to provide compositions that exhibit the required film forming and processing characteristics for uniform and non-agglomerating film coating of pellets. The inventors have surprisingly found, that a combination of excipients with the following functional properties produce well-formed (coated) pellets using electrostatic powder coating processes. These functional properties include film forming polymers for the intended release characteristics, plasticizers for optimal film forming temperature, anti-static agents for charge distribution, and flow aids for metering powders for coating.

One surprising finding of these powder coatings is that the powder coating formulations can be prepared in a pan coater. Because of the difficulties in coating pellets compared to tablets, the liquid coating of pellets are generally produced using a fluidbed with Wurster inserts, see U.S. Pat. No. 3,241,520 (Wruster 1966) which shows a bottom sprayed fluidized bed with a Wurster insert.

### Film Forming Polymers

Film forming polymers that can achieve, immediate release, flavoring or taste modifying/masking or moisture barrier include, but are not limited to, any one or combination of methylcellulose, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers such as Eudragit® E.

Coating polymers that could achieve extended release include, but not limit to a cellulose ether derivative, an acrylic resin, a copolymer of acrylic acid and methacrylic acid esters with quaternary ammonium groups, a copolymer of acrylic acid and methacrylic acid esters or a combination of any thereof, or it can include ethyl cellulose, cellulose acetate, poly(meth)acrylates polymers that are not soluble in digestive fluids such as Eudragit® RS and RL polymers with alkaline groups and EUDRAGIT® NE polymers with neutral groups.

Coating polymers that exhibit extended release include water soluble polymers such as, but not limit to, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like. Coating polymers that could achieve delayed release include, but are not limited to, any one or combination of cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups (such as Eudragit® L30D, Eudragit® L100, Eudragit® FS30D, Eudragit® SI00, Acryl-EZE®).

It will be appreciated by those skilled in the art that multiple coats may be applied to the pellets with each coat selected to have a pre-determined functionality as set out above with respect to the film forming polymers.

### Plasticizers

Both liquid and solid plasticizers can be used to achieve the target glass transition temperature for powder coating, and may be present in the composition in quantity to lower the glass transition temperature of the coating composition to broadly in the temperature range from about 30 to 100° C. and more preferably from 45 to 70° C. It has been surprisingly found that the liquid plasticizers have multiple functions in the present pellet coatings. The functions of the plasticizers used include: 1) lowering the glass transition temperature (i.e., increase in molecular mobility) of the film forming polymer(s) to produce satisfactory functional or cosmetic coating for oral pharmaceutical formulations; 2) increased adhesion of the film forming powder to the pellet substrate; and 3) increasing the electrical conductivity on spraying the substrate surface during coating. Thus the surface plasticizer also acts as an anti-static agent before it is incorporated into coating polymer matrix to produce a polymer film.

The plasticizers can be incorporated with the chain of the main formulation of the film forming coating powder, as a result, the free volume between polymer chains can be increased and the glass transition temperature of the polymer powder can be reduced dramatically. When the plasticizer is comprised of liquid polymers or polymer solutions, a certain amount of the plasticizer on the surface of the pellets can also decrease the electrical resistance of the pellets dramatically so that the adhesion of charged coating powder and the coating uniformity and efficiency is improved. Furthermore, a certain amount of liquid plasticizer or plasticizer solution can provide a strong capillary force between particles and allow polymer sintering and film formation to occur.

Plasticizers suitable for use in the present coating formulations include, but are not limited to, glycerol, propylene glycol, PEG 200-600 grades, triacetin, diethyl phthalate (DEP), dibutyl phthalate (DBP) and tributyl citrate (TBC), triethyl citrate (TEC), castor oil, fractionated coconut oil, acetylated monoglycerides and glycerol monostearate.

Plasticizers suitable for use in the present invention also include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, and citrate ester-type plasticizers. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, acetyl triethyl citrate, tributyl citrate and allyl glycolate.

### Anti-Static Agents

The one or more anti-static agents may include common salts, carbon black, magnesium stearate, fumed silicate, magnesium trisilicate, glycerol monostearate, Kaolin, talc and a liquid plasticizer. The liquid plasticizer may include any one or combination of PEG 200 to 600, propylene glycol, glycerin, and triacetin. The common salts may include, but are not limited to, any one or combination of sodium chloride, calcium chloride, magnesium hydroxide, sodium carbonate, sodium bicarbonate, sodium phosphate, sodium citrate, sodium acetate, potassium acetate, potassium citrate, potassium chloride, and magnesium sulfate. The anti-static agents may be present in the composition in a range from about 0.1 to about 95% w/w, and more preferably in a range from about 1 to about 50% w/w.

### Flow Enhancing Agents

The one or more flow enhancing agents may include any one or combination of calcium stearate, colloidal silicon dioxide, hydrogenate castor oil and microcrystalline cellulose, fumaric acid, glycerol behanate, glycerol monostearate, glycerol palmitostearate, leucine, magnesium stearate, medium chain triglyceride, myristic acid, palmitic acid, poloxamer, polyethylene glycol, potassium benzoate, sodium benzoate, sodium lauryl sulfate, sodium stearyl fumarate, starch, stearic acid, talc, hydrogenated vegetable oil and zinc stearate.

The one or more flow enhancing agents in powder form may be present in the composition in a range from about 0.1 to about 25% w/w, and more preferably from about 0.25 to about 20% w/w. In embodiments the one or more flow enhancing agents is present in the composition in a range from about 0.5 to about 3% w/w.

In an embodiment a powder coating composition for pharmaceutical pellets, comprises:
a) one or more film forming polymers in powder form present in the composition in a range from about 1 to about 95% w/w;
b) one or more plasticizers in powder or liquid form present in the composition in quantity to lower the glass transition temperature of the coating composition to a temperature in a range from about 30 to 100° C.;
c) one or more anti-static agents in powder or liquid form present in the composition in a range from about 0.1 to about 95% w/w; and
d) one or more flow enhancing agents in powder form present in the composition in a range from about 0.1 to about 25% w/w.

In an embodiment the one or more film forming polymers is present in the composition in a range from about 10 to about 70% w/w.

In an embodiment the one or more flow enhancing agents is present in the composition in a range from about 0.25 to about 20% w/w.

In an embodiment the one or more flow enhancing agents is present in the composition in a range from about 0.5 to about 3.0% w/w.

In an embodiment the one or more anti-static agents are present in the composition in a range from about 1 to about 50% w/w.

In an embodiment the one or more plasticizers include any one or combination of glycerol, propylene glycol, PEG 200 to 8000 grades, triacetin, diethyl phthalate (DEP), dibutyl phthalate (DBP), tributyl citrate (TBC), triethyl citrate (TEC), castor oil, fractionated coconut oil, acetylated monoglycerides, glycerol monostearate, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol) and citrate ester-type plasticizers.

In an embodiment the one or more plasticizers include any one or combination of ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, acetyl triethyl citrate and allyl glycolate.

In an embodiment the one or more anti-static agents include common salts, carbon black, magnesium stearate, fumed silicate, magnesium trisilicate, glycerol monostearate, Kaolin, talc and a liquid plasticizer.

In an embodiment the liquid plasticizer includes any one or combination of PEG 200 to 600, propylene glycol, glycerin, and triacetin.

In an embodiment the common salts include any one or combination of sodium chloride, calcium chloride, magnesium hydroxide, sodium carbonate, sodium bicarbonate, sodium phosphate, sodium citrate, sodium acetate, potassium acetate, potassium citrate, potassium chloride, and magnesium sulfate.

In an embodiment the plasticizer is selected to lower the glass transition temperature of the coating composition to a temperature in a range from about 45 to 70° C.

In an embodiment the one or more flow enhancing agents include any one or combination of calcium stearate, colloidal silicon dioxide, hydrogenate castor oil and microcrystalline cellulose, fumaric acid, glycerol behanate, glycerol monostearate, glycerol palmitostearate, leucine, magnesium stearate, medium chain triglyceride, myristic acid, palmitic acid, poloxamer, polyethylene glycol, potassium benzoate, sodium benzoate, sodium lauryl sulfate, sodium stearyl fumarate, starch, stearic acid, talc, hydrogenated vegetable oil and zinc stearate.

In an embodiment the one or more film forming polymers is selected to exhibit any one or combination of a moisture barrier, immediate release, flavoring, taste modifying, and taste masking, and wherein the film forming polymer includes any one or combination of methylcellulose, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers.

In an embodiment the one or more film forming polymers is selected to exhibit extended release and includes any one or combination of cellulose ether derivative, acrylic resin, a copolymer of acrylic acid and methacrylic acid esters with quaternary ammonium groups, a copolymer of acrylic acid and methacrylic acid esters, ethyl cellulose, and poly(meth)acrylate polymers that are not soluble in digestive fluids.

In an embodiment the poly(meth)acrylate polymers that are not soluble in digestive fluids include any one or combination of Eudragit® RS polymers, Eudragit® RL polymers, and EUDRAGIT® NE polymers.

In an embodiment the one or more film forming polymers is selected to exhibit extended release and includes any one or combination of polyethylene oxide (PEO), ethylene oxide-propylene oxide co-polymers, polyethylene-polypropylene glycol (e.g. poloxamer), carbomer, polyvinyl pyrrolidone (PVP), polyvinyl alcohol (PVA), hydroxyalkyl celluloses such as hydroxypropyl cellulose (HPC), hydroxypropyl methylcellulose, sodium carboxymethyl cellulose, methylcellulose, hydroxyethyl methylcellulose, hydroxypropyl methylcellulose, polyacrylates such as carbomer, polyacrylamides, alginic acid and its derivatives, starch and starch derivatives, gelatin that are soluble in digestive fluids.

In an embodiment the one or more film forming polymers is selected to exhibit delayed release include any one or combination of cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups.

In an embodiment the methacrylic copolymers with carboxylic acid groups include Eudragit® L30D, Eudragit® L100, Eudragit® FS30D, Eudragit® SI00, and Acryl-EZE®.

In an embodiment the compositions above are applied multiple times to the pellets with each different coating selected to have a pre-determined functionality.

Specifically, the multilayer coated pellets with each layer including a predetermined composition can provide extra clinical benefits, leading to a multifunctional particulate (pellet) drug delivery system that capable of achieving a variety of drug release profiles, such as instant drug release with a moisture barrier and drug protection, sustained/controlled drug release and delayed drug release. This multifunctional particulate (pellet) drug delivery system could not only have drug pellets (containing APIs) coated with different coating compositions, but also could have one or more drug loaded onto the surface of the pellet cores (such as sugar based pellets), then further coated with predesigned coating compositions.

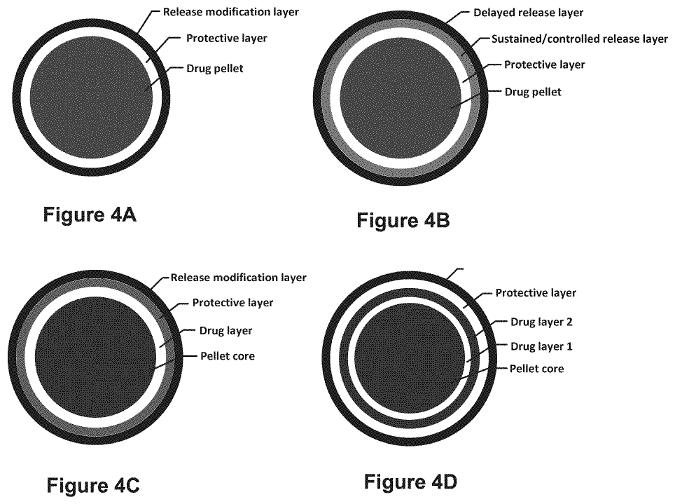

FIGS. 4A to 4D illustrate examples of multilayer coated pellets with different coating compositions. FIG. 4A shows an example of drug pellet pre-coated with the protective layer containing protective compositions above to protect the drug pellet. Non-limiting examples of the constituents of this protective composition include, but are not limited to, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers, or any mixture or combination thereof.

Then the pre-coated pellets are coated with release modification compositions to achieve delayed release or sustained/controlled release layer or any other modified release profiles. Non-limiting examples of the constituents of these release modification compositions include, but are not limited to, water soluble, water insoluble and pH sensitive polymers such as, but not limit to, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups (such as Eudragit® L30D, Eudragit® L100, Eudragit® FS30D, Eudragit® SI00, Acryl-EZE®) or any mixture or combination thereof.

FIG. 4B shows an example of a drug pellet pre-coated with the protective layer containing protective compositions to protect drug from being damaged during the following coating process. Non-limiting examples of the constituents of this protective composition include, but are not limited to, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers, or any mixture or combination thereof.

The protective layer can also include the following additional constituents including one of more acidic or basic organic or inorganic salts or pH modifiers such as but not limited to, ammonium hydroxide, alkali metal salts, alkaline earth metal salts such as but not limited to, ammonium hydroxide, alkali metal salts, alkaline earth metal salts such as but not limited to, ammonium hydroxide, alkali metal salts, alkaline earth metal salts such as potassium and sodium potassium and sodium potassium and sodium phosphate, sodium and potassium acetate, magnesium oxide, magnesium hydroxide, calcium hydroxide, sodium hydroxide, potassium hydroxide, aluminum hydroxide, potassium carbonate, sodium bicarbonate; an hygroscopic agents such as but not limited to, ammonium hydroxide, alkali metal salts, alkaline earth metal salts such as potassium and sodium calcium chloride, antioxidants, such as but not limited to, ammonium hydroxide, alkali metal salts, alkaline earth metal salts such as ascorbic acid, EDTA, potassium and sodium butylated hydroxytoluene and butylated hydroxyanisole; physical or chemical complex forming agents such as, but not limited to, polyvinyl pryrrolidine or cross linked polyvinylpolypyrrolidone.

A second layer on the protective layer coatings is a sustained/controlled release layer and then delayed release layer, achieving both delayed drug release and sustained/controlled release. Non-limiting examples of the constituents of these sustained/controlled release and delayed release modification compositions include, but are not limited to, water soluble, water insoluble and pH sensitive polymers such as, but not limit to, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups (such as Eudragit® L30D, Eudragit® L100, Eudragit® FS30D, Eudragit® SI00, Acryl-EZE®), or any mixture or combination thereof.

FIG. 4C shows an example of a pellet core (for instance, sugar based pellets) firstly loaded with a single drug layer, Non-limiting examples of the constituents of these drug compositions include, but are not limited to, e.g., moisture sensitive drugs such as aspirin, melbine, esomeprazole, vitamins and so on; anti-inflammatory, antipyretic, anticonvulsant and/or analgesic agents such as indomethacin, nimesulide, ibuprofen, fenoprofen calcium, etc; cardiocirculatory system drugs such as nifedipine, felodipine, nimodipine, nilvadipine, lacidipine, doxazosin and anti-asthma drugs such as salbutamol. Others having the same or different physiological activity as those above, or suitable mixture thereof, can also be employed in accordance with the present disclosure.

This first drug layer coated directly on the pellet is then coated by the protective coating layer containing protective compositions to protect drug from being damaged during the following coating process. Non-limiting examples of the constituents of this protective composition include, but are not limited to, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers or any mixture or combination thereof. The release modification coating layer is then applied over this protective composition to achieve delayed release or sustained/controlled release layer or any other modified release profiles. Non-limiting examples of the constituents of these sustained/controlled release and delayed release modification compositions include, but are not limited to, water soluble, water insoluble and pH sensitive polymers such as, but not limit to, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups (such as Eudragit® L30D, Eudragit® L100, Eudragit® FS30D, Eudragit® SI00, Acryl-EZE®), or any mixture or combination thereof.

FIG. 4D is an example of a pellet core coated with two different drug layers, a first layer located directly onto the pellet and second drug layer coated on the first layer. Non-limiting examples of the constituents of the two drug layer compositions include, but are not limited to, e.g., moisture sensitive drugs such as aspirin, melbine, esomeprazole, vitamins and so on; anti-inflammatory, antipyretic, anticonvulsant and/or analgesic agents such as indomethacin, nimesulide, ibuprofen, fenoprofen calcium, etc; cardiocirculatory system drugs such as nifedipine, felodipine, nimodipine, nilvadipine, lacidipine, doxazosin and anti-asthma drugs such as salbutamol, or any combination thereof. Others having the same or different physiological activity as those above, or suitable mixture thereof, can also be employed in this invention.

A protective coating layer is then deposited onto the top surface of the second drug layer with the protective layer containing protective compositions on the second drug coating followed by a release modification layer to achieve delayed release or sustained/controlled release layer or any other modified release profiles. The protective layer contains protective compositions to protect drug from being damaged during the following coating process. Non-limiting examples of the constituents of this protective composition include, but are not limited to, hydroxyethyl cellulose, hydroxypropyl cellulose (HPC), hydroxylpropyl methyl cellulose (HPMC), polyethylene glycol, propylene glycol, polaxamer and povidone, polyvinyl alcohol based composition such as Opadry® AMB, Aminoalkyl methacrylate copolymers, or any combination thereof.

Non-limiting examples of the constituents of these sustained/controlled release and delayed release modification compositions include, but are not limited to, water soluble, water insoluble and pH sensitive polymers such as, but not limit to, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin, cellulose acetate phthalate, cellulose acetate trimaletate, hydroxyl propyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, methacrylic copolymers with carboxylic acid groups (such as Eudragit® L30D, Eudragit® L100, Eudragit® FS30D, Eudragit® SI00, Acryl-EZE®), or any combination thereof.

Several non-limiting examples are given below.

### EXAMPLES

In the present examples, piroxicam pellets were used as the model drug coating pellets to demonstrate the effectiveness of the electrostatic powder coating compositions provided in the present disclosure. Three different classes of functional pharmaceutical polymers compositions containing Eudragit® EPO, Eudragit® RS/RL, Acryl-EZE®, were selected to achieve taste masking, extended release and delayed release, respectively.

### Example 1

Dry Powder Coating of Piroxicam Pellets with a Taste Masking Coating (Eudragit® EPO)

This example demonstrates the dry powder coating of piroxicam pellets using a coating composition (Table 1) containing Eudragit® EPO (a cationic copolymer based on dimethylaminoethyl methacrylate, butyl methacrylate, and methyl methacrylate), a pH sensitive polymer that is soluble in gastric juice up to pH 5.0, swellable and permeable above pH 5.0, and a liquid plasticizer, polyethylene glycol 400 (PEG 400, EMD Chemicals Inc. Ontario, Canada), is used to increase the adhesion between the coating powder and the piroxicam pellets. Talc is used as the anti-static agent and colloidal silicon dioxide is used as the flow enhancing agent.

### Preparation of the Coating Powder

The coating powder was prepared using a blade grind mill for about 25 seconds following the composition shown in Table 1. Eudragit® EPO and colloidal silicon dioxide (AEROSIL 200® Pharma) were donated by Evonik Degussa Corporation (Germany). Talc was purchased from Mallinckrodt Baker Inc. (Canada).

TABLE 1
Composition of taste masking coating powder*

| Formulation | Composition (% w/w) |
|---|---|
| Eudragit ® EPO | 10.0 |
| Talc | 89.0 |
| Colloidal silicon dioxide(nano level) | 0.5 |
| Pigment(FD&C Yellow No. 6) | 0.5 |

*The plasticizer, PEG 400, is included in the coating composition by spraying onto the coating pellets. The particle size (volume mean diameter) D[4,3] of the above used Eudragit ® EPO and Talc powder are 13.3 μm and 28.9 μm, respectively.

### 1) Powder Coating Process

40 g piroxicam pellets were loaded into the rotatable drum of a rotary powder coating apparatus and was pre-heated to 40° C. at a rotating speed of 20 rpm. Then the rotation speed of the drum was increased to 70 rpm and the temperature was maintained at 40° C. Liquid plasticizer (PEG 400) was sprayed on to the particles from an atomizing spraying nozzle at a flow rate of 0.25 g/min for 35 seconds. 1.5 g coating materials were immediately deposited to the coating particles after plasticizer spraying. The plasticizer spraying and coating materials deposition cycle was repeated after about 15 mins 3 times until the target coating level was achieved. The particles were cured at 40° C. and a rotating speed of 20 rpm for 2 hours.

### 2) Dissolution Test

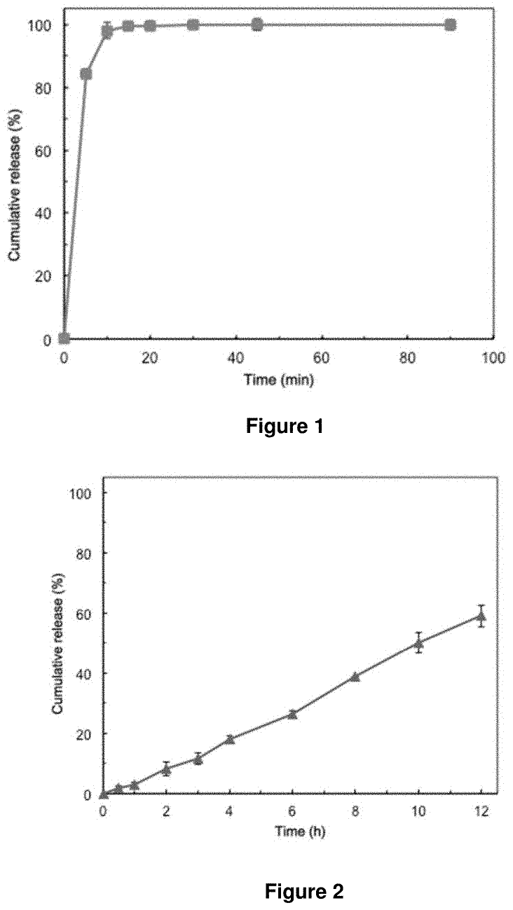

The coated piroxicam pellets was visually examined in phosphate buffer solution (PBS) at pH 6.8. No dissolution was observed and the film coat was intact for up to 10 minutes. The dissolution profile of coated piroxicam pellets in 0.1 N HCl solution (pH=1.2) was obtained using an USP dissolution apparatus (Apparatus 2) at 37° C. and a rotation speed of 100 rpm. The dissolution samples were assayed using a UV-Vis spectrophotometer at a wavelength of 334 nm. The PBS and rapid dissolution in 0.1N HCl results shown in FIG. 1 indicate that the coated pellets exhibit taste masking behavior, i.e. little or no dissolution upon swallowing and rapid dissolution when the product reaches the stomach.

### Example 2

Dry Powder Coated Piroxicam Pellets with Extended Release Coating (Eudragit® RS/RL)

This example demonstrate the dry powder coating of piroxicam pellets using a coating composition (Table 2) containing Eudragit® RS (a low permeability copolymer of ethyl acrylate, methyl methacrylate and a low content of methacrylic acid ester with quaternary ammonium groups.) and Eudragit® RL (a high premeabillity copolymer of ethyl acrylate, methyl methacrylate and a low content of methacrylic acid ester with quaternary ammonium groups.), two pH independent polymers that are commonly used for extended release coating. A liquid plasticizer, triethyl citrate (TEC, Caledon Laboratories Ltd. Ontario, Canada), is also used to increase the adhesion between the coating powder and the piroxicam pellets and to decrease the T_{g} of the Eudragit® RS/RL from 63-65 to around 35° C. Talc is used as the anti-static agent and colloidal silicon dioxide is used as the flow enhancing agent.

### Preparation of the Coating Powder

The coating powder was prepared using a blade grind mill for about 25 seconds following the composition shown in Error! Reference source not found. Eudragit® RS and Eudragit® RL and colloidal silicon dioxide (AEROSIL 200® Pharma) were donated by Evonik Degussa Corporation (Germany). Talc was purchased from Mallinckrodt Baker Inc. (Canada).

TABLE 2
Composition of extended release coating powder*

| 1) Formulation | Composition (% w/w) |
|---|---|
| Eudragit ® RS | 40.0 |
| Eudragit ® RL | 40.0 |
| Talc | 19.0 |
| Colloidal silicon dioxide (nano level) | 0.5 |
| Pigment(FD&C Blue number 1) | 0.5 |

*A liquid plasticizer, TEC, is included in the coating composition by spraying onto the coating pellets.
The particle size (volume mean diameter) D[4,3] of the above used Eudragit ® RS, Eudragit ® RL and Talc were 47.7 μm, 40.8 μm and 28.9 μm, respectively.

### 2) Powder Coating Process

40 g piroxicam pellets were loaded into the rotatable drum of a rotary powder coating apparatus and was pre-heated to 50° C. at a rotating speed of 20 rpm. Then the rotation speed of the drum was increased to 70 rpm and the temperature was maintained at 50° C. Liquid plasticizer (TEC) was sprayed on to the particles from an atomizing spraying nozzle at a flow rate of 0.25 g/min for 35 seconds. 1.5 g coating materials were immediately deposited to the coating particles after plasticizer spraying. The plasticizer spraying and coating materials deposition cycle was repeated after about 15 mins 6 times until the target coating level was achieved. The particles were cured at 50° C. and a rotating speed of 20 rpm for 2 hours.

### 3) Dissolution Test

The dissolution profile of coated piroxicam pellets in pH=7.0 phosphate buffer solution was obtained using an USP dissolution apparatus (Apparatus 2) at 37° C. and a rotation speed of 50 rpm. The dissolution samples were assayed using a UV-Vis spectrophotometer at a wavelength of 354 nm. FIG. 2 is the dissolution profile of the coated piroxicam pellets with Eudragit® RS/RL which demonstrated the expected extended release function of the coated formulation.

### Example 3

Dry Powder Coated Piroxicam Pellets with Delayed Release Coating (Acryl-EZE®)

This example demonstrates the dry powder coating of piroxicam pellets using a enteric coating composition (Table 3) containing Acryl-EZE® (contains Eudragit® L100-55, an anionic copolymer based on methacrylic acid and ethyl acrylate provided by Colorcon Inc. USA), a formulated coating pH sensitive coating powder that is soluble in water at a pH above 5.5.

A liquid plasticizer, polyethylene glycol 400 (PEG 400, EMD Chemicals Inc. Ontario, Canada), is used to increase the adhesion between the coating powder and the piroxicam pellets and to decrease the T_{g} of the Acryl-EZE® from 133° C. to 50-55° C. The plasticizer also serves as an anti-static agent.

### 1) Preparation of the Coating Powder

The coating powder was prepared using a blade grind mill for about 25 seconds following the composition shown in Table 2.

TABLE 2
Composition of delayed release coating materials*

| Formulation | Composition (% w/w) |
|---|---|
| Acryl-EZE | 99.5 |
| pigment (FD&C Blue number 1) | 0.5 |

*The plasticizer, PEG 400, is included in the coating composition by spraying onto the coating pellets. The particle size (volume mean diameter) D[4,3] of the above used Acryl-EZE ® was 20.5 μm.

### 2) Powder Coating Process

40 g piroxicam pellets were loaded into the rotatable drum of a rotary powder coating apparatus and was pre-heated to 50° C. at a rotating speed of 20 rpm. Then the rotation speed of the drum was increased to 70 rpm and the temperature was maintained at 50° C. Liquid plasticizer (PEG 400) was sprayed on to the particles from a atomizing spraying nozzle at a flow rate of 0.25 g/min for 35 seconds. 1.5 g coating materials were immediately deposited to the coating particles after plasticizer spraying. The plasticizer spraying and coating materials deposition cycle was repeated after about 15 mins for several times (4 times for coating level of 13.25% w/w; 7 times for 21.93% w/w) until the target coating level was achieved. The particles were cured at 50° C. and a rotating speed of 20 rpm for 2 hours.

### 3) Dissolution Test

The dissolution profile of coated piroxicam pellets was obtained in 0.1 N (pH=1.2) HCl solution for 2 hours (acid stage) and in pH=6.8 phosphate buffer solution after the acid stage using an USP dissolution apparatus at 37° C. and at a rotation speed of 100 rpm. The dissolution samples were assayed using a UV-Vis spectrophotometer at a wavelength of 334 nm (acid stage samples) and 353 nm (buffer stage samples).

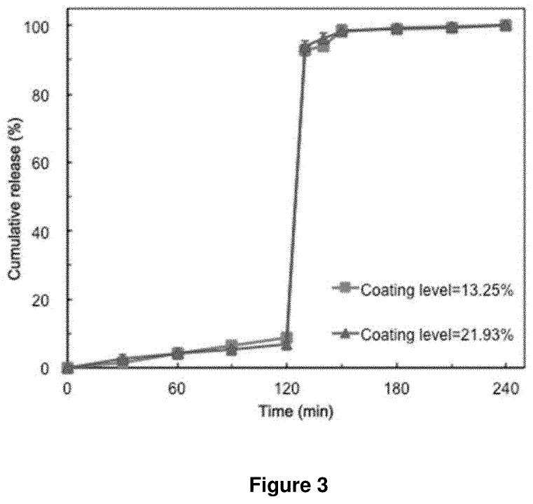

The delayed release profiles of the coated pellets at coating level of 13.25 and 21.93% w/w are shown in FIG. 3. In both cases, the results met and exceeded the requirements of the acid resistance test of percent release of not more than 10% released in 0.1 N HCl in 2 hours.

### Example 4

Dry Powder Coated Esomeprazole Pellets with Dry Layering, Protective Coating and Extended Release Coatings

This example demonstrates the dry powder coating of esomeprazole pellets in multilayers using three coating compositions (Table 4) applied in sequence onto nonpareil pellets such as sugar based pellets. The first, second and third coating layers provide immediately release, chemical degradation protection and immediate release, and enteric release characteristics, respectively, upon dissolution.

The first layer of coating contains esomeprazole (the drug substance), hydroxypropyl cellulose (HPC) (a pH independent and water soluble polymer) and PVP-XL (crosslinked polyvinylpyrrolidone) and magnesium hydroxide (two stabilizing agents), talc (an anti-static agent) and polyethylene glycol 400 (PEG 400) (a plasticizer and an agent to improve adhesion of the coating powder onto the pellets).

The second layer of coating contains, hydroxypropyl cellulose (HPC) (a pH independent and water soluble polymer), PVP-XL (crosslinked polyvinylpyrrolidone) and magnesium hydroxide (two stabilizing agents), talc (an anti-static agent) and polyethylene glycol 400 (PEG 400) (a plasticizer and an agent to improve adhesion of the coating powder onto the pellets).

The third layer of coating contains Eudragit® L100-55 (an anionic copolymer based on methacrylic acid and ethyl acrylate, a pH sensitive coating powder that is soluble in water at a pH above 5.5), talc (an anti-static agent) and polyethylene glycol 400 (PEG 400) (a plasticizer and an agent to improve adhesion of the coating powder onto the pellets).

TABLE 3
Composition of drug layering, protective
and extended release coating materials*

Composition (% w/w)

Drug Layering Formulation
(First coating layer)

| | |
|---|---|
| Esomeprazole | 60.0% |
| HPC | 20.0% |
| PVP-XL | 5.0% |
| Magnesium hydroxide | 10.0% |
| Talc | 5.0% |

Protective Layer Formulation
(Second coating layer)

| | |
|---|---|
| HPC | 60.0% |
| PVP-XL | 20.0% |
| Magnesium hydroxide | 10.0% |
| Talc | 10.0% |

Delayed Release Layer Formulation
(Third coating layer)

| | |
|---|---|
| Eudragit ® L100 55 | 60.0% |
| Talc | 40.0% |

*A liquid plasticizer, PEG400 is included in the coating composition by spraying onto the coating pellets.

### Preparation of the Coating Powder

The particle size of HPC and Eudragit L100 55 are reduced to 30 um and 23 um, respectively, using an air jet mill. Then the coating compositions excepted for PEG 400 were mixed before use.

### Powder Coating Process

Sucrose based pellets were loaded into the rotatable drum of a rotary powder coating apparatus and was pre-heated to 45° C. at a rotation speed of 20 rpm. The rotation speed of the drum was increased to about 70 rpm and the temperature was maintained at 45° C. Liquid plasticizer (PEG 400) was sprayed on to the particles from an atomizing spraying nozzle at a flow rate of approximately 0.25 g/min for 35 seconds. 1.5 g coating materials were immediately deposited to the coating particles after plasticizer spraying. The plasticizer spraying and coating materials deposition cycle was repeated after every 15 mins until the target coating level was achieved. The same process was repeated for subsequent coating layers. The coated pellets were cured at 45° C. for up to 72 hours.

### 4) Dissolution Test

The dissolution profile of coated esomeprazole pellets in pH=1.0 buffer solution (for 2 hours) and in pH 6.8 buffer solution (for another 2 hours) was obtained using an USP dissolution apparatus (Apparatus 2) at 37° C. and a rotation speed of 100 rpm. The dissolution samples were assayed using a UV-Vis spectrophotometer at a wavelength of 305 nm for the pH 1.0 and 280 nm for the pH 6.8 samples. The dissolution results confirm that the enteric coating met the requirements of the USP.

The coated esomeprazole pellets were placed in sealed glass containers and stored at 50° C. for up to 6 weeks. No change of color of the pellets was observed. The results indicate that the stabilizing agents in the coating layers are effective in preventing or reducing esomeprazole degradation. Also the protective coating layer is providing an effective barrier in preventing the chemical interaction (chemical degradation) between the incompatible components, esomeprazole and Eudragit® L100-55, in the coated pellets.

All of the formulation compositions can be made and executed without undue experimentation in light of the present disclosure. While the formulation compositions, methods of this disclosure have been described in terms of preferred embodiments, it will be apparent to those of skill in the art that variations may be applied to the formulation compositions, and/or apparatus and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

Therefore what is claimed is:

1. A powder coating composition for pharmaceutical pellets, comprising:
a) one or more film forming polymers in powder form present in the composition in a range from about 1 to about 95% w/w;
b) one or more plasticizers in powder or liquid form present in the composition in quantity to lower the glass transition temperature of the coating composition to a temperature in a range from about 30 to 100° C.;
c) one or more anti-static agents in powder or liquid form present in the composition in a range from about 0.1 to about 95% w/w; and
d) one or more flow enhancing agents in powder form present in the composition in a range from about 0.1 to about 25% w/w.
